(19) United States
(12) Patent Application Publication
(10) Pub. No.: US 2013/0123905 A1
(43) Pub. Date: May 16, 2013

(54) OFFSET PEAK-TO-PEAK STENT PATTERN

(71) Applicant: ABBOTT CARDIOVASCULAR SYSTEMS INC., Santa Clara, CA (US)

(72) Inventors: Chad J. Abunassar, San Francisco, CA (US); Tanya B. Schikorr, Mountain View, CA (US); Laura M. Kalvass, Mountain View, CA (US); Erik D. Eli, Redwood City, CA (US); Diem U. Ta, San Jose, CA (US)

(73) Assignee: ABBOTT CARDIOVASCULAR SYSTEMS INC., Santa Clara, CA (US)

(21) Appl. No.: 13/628,339

(22) Filed: Sep. 27, 2012

Related U.S. Application Data
(60) Provisional application No. 61/560,071, filed on Nov. 15, 2011.

(51) Int. Cl. A61F 2/06

(52) U.S. Cl. 623/1.16

(57) ABSTRACT

The invention is directed to an expandable stent for implanting in a body lumen, such as a coronary artery, peripheral artery, or other body lumen. The invention provides for an intravascular stent having a plurality of cylindrical rings connected by links. The links between adjacent rings provide axial strength when subjected to longitudinal compressive forces.

### CROSS-REFERENCES TO RELATED APPLICATIONS

This application claims priority to U.S. Ser. No. 61/560,071 filed Nov. 15, 2011, the entire contents of which are incorporated herein by reference.

### BACKGROUND

The invention relates generally to intravascular stents for use in the coronary arteries and other body lumens of human patients.

Stents are generally tubular-shaped devices which function to hold open a segment of a blood vessel or other body lumen such as a coronary artery. They also are suitable for use to support and hold back a dissected arterial lining that can occlude the fluid passageway. At present, there are numerous commercial stents being marketed throughout the world. For example, the prior art stents depicted in FIGS. 1-5 typically have multiple cylindrical rings connected by one or more connecting links. While some of these stents are flexible and have the appropriate radial rigidity needed to hold open a vessel or artery, there typically is a tradeoff between flexibility and radial strength and the ability to tightly compress or crimp the stent onto a catheter so that it does not move relative to the catheter or dislodge prematurely prior to controlled implantation in a vessel.

Intravascular stents are well known and there are numerous structural designs in commercial use. One well known structural pattern includes a tubular stent having rings connected by links. Typically, there are two or more links connecting adjacent rings. While stents having two links between adjacent rings (two-link stents) offer the benefit of low crimp profile and high flexibility, these benefits come with a trade-off in terms of longitudinal stability. Further, peak-to-peak stent patterns (in which the peaks on adjacent rings point toward each other and are essentially axially aligned) offer dense packing of stent rings, which in turn allows for a stent pattern with high radial strength and high radial stiffness. One stent pattern that incorporates these design features is the 2 link offset peak-to-peak style stent. While this stent pattern performs well in terms of traditional stent metrics, it experiences one key tradeoff, namely it will excessively shorten under modest longitudinal compressive loads.

Two-link stents, specifically offset peak-to-peak, where the peaks of adjacent rings point toward each other but are slightly offset circumferentially, excessively shorten under modest (clinically relevant) longitudinal compressive loads. This creates unwanted implications for safety and efficacy of the stent implant. Specific reasons identified for this longitudinal instability and/or poor longitudinal stiffness are complex as set forth below:
(1) Insufficient number of links to bear a clinically relevant longitudinally compressive load;
   (a) Two-link designs provide only two paths for longitudinal load to react through the stent structure.
   (b) Sub-optimal placement of load-bearing links.
(2) Excessively unsupported ring structure deforms easily under longitudinally compressive loads;
   (a) Substantial unsupported ring structure exists between links in a specific ring (cantilever effect);
   (b) Unevenly or misaligned expanded stent structure encourages adjacent rings/crests to nest within each other without experiencing any substantial resistance until the structure has compressed excessively;
   (c) The combination of both above reasons under heading (2) exacerbates longitudinal instability and produces substantial nesting as shown on a compressed stent (see the prior art stent of FIG. 1).
(3) Offset and angled link designs lend readily to collapse behavior, as links do not provide resistance in direction of load;
   (a) Offset link designs create a bending moment effect, which encourages the bar arms adjacent to link structures to bend and swing excessively (stress is focused in these bar arms as shown in FIG. 4);
   (b) Offset link designs create a structure that does not experience strut-to-strut contact during longitudinal compression; other peak-to-peak designs exhibit increased longitudinal stiffness by reacting load between adjacent rings during strut-to-strut contact; however, this is not the case for offset peak-to-peak designs;
(4) Peak-to-peak patterns inherently shorten when expanded; if this shortening is prevented due to balloon growth or friction, the structure may retain residual stress that encourages a sudden shortening behavior under longitudinal compression loads. (This aspect may play more of a role in super-elastic stents).

The uniquely poor behavior of some offset peak-to-peak commercially available stent patterns has been studied, where test results have shown that under clinically relevant compressive loads (50 grams of force), the Element stent (manufactured by Boston Scientific) having a two-link offset peak-to-peak stent pattern exhibited excessive shortening while all other stent patterns studied provided substantially more compressive resistance and stability. Several commercially available stent patterns are illustrated in FIGS. 2-5 and the test method and results are described.

In FIG. 2, the Multi-Link Vision stent (Abbott Cardiovascular Systems Inc.) has an in-phase, peak-to-valley ring pattern and three links connecting adjacent rings from the valley of a crest on one ring to the peak of a crest on the adjacent ring.

In FIG. 3, the Endeavor Sprint stent (Medtronic, Inc.) has an out-of-phase peak-to-peak ring pattern and the connection between two adjacent rings occurs from the peak of a crest on one ring to the peak of a crest on the adjacent ring.

In FIG. 4, the Cypher Select Plus stent (Johnson & Johnson) has adjacent rings connected by a mid-strut connector. The links connecting adjacent rings connect from the strut of one ring to the strut of the adjacent ring. A strut is a geometric element of the sinusoidal ring that connects adjacent crests on a particular ring.

In FIG. 5, the Element stent (Boston Scientific) has an offset peak-to-peak ring pattern. The links between two adjacent rings occurs from the peak of a crest on one ring to the peak of a crest on the adjacent ring and the link is a geometric element that is positioned at an angle.

Offset link designs (e.g., Element stent) create a bending moment effect, which induces bar arms adjacent to the links to incur bending stresses/deformation while also encouraging nesting between adjacent rings. Further, the offset link design creates a structure that does not experience strut-to-strut contact during longitudinal compression. Non-offset peak-to-peak designs exhibit increased longitudinal stiffness by redirecting the load between adjacent rings through strut-to-strut contact (e.g., Medtronic Driver stent); however, this is not the case for offset peak-to-peak stents (e.g., Boston Scientific Element stent).

The testing procedure conducted on a number of commercially available stents is shown in FIGS. 6A and 6B. As shown in FIG. 6A, an expanded stent was placed on a mandrel and then mounted in a longitudinal compression fixture. As shown in FIG. 6B, an incremental longitudinal compressive load was applied to the stents being tested, with the maximum longitudinal compression being of about 50 grams of force and longitudinal shortening of about 14 mm. The test results are shown in the graph in FIG. 7. The stents having links connecting between peak-to-valley, peak-to-peak, and mid-strut, all exhibited comparable and acceptable bending stresses and deformation under the longitudinal compressive loads (see, e.g., FIG. 8A). Only the offset peak-to-peak link connection between adjacent rings of the Element stent (Boston Scientific) exhibited unacceptable bending stresses and deformation (see FIG. 8B) including undesirable strut-to-strut contact.

The physical test results were supported by finite element analysis (FEA) conducted on offset peak-to-peak stents to determine the longitudinal compression behavior of the stent pattern. The uncompressed image (FIG. 9A) of an expanded offset peak-to-peak stent shows some deformation, however, the compressed image (FIG. 9B) shows substantial localized bending stresses and deformation in the bar arms adjacent to the stent links, as well as undesirable nesting of rings within adjacent rings without substantial strut-to-strut contact.

What has been needed is a stent pattern that exhibits good column strength when subjected to longitudinal compressive loads yet remains longitudinally flexible so that the stent easily navigates tortuous coronary arteries and other body lumens.

### SUMMARY OF THE INVENTION

The present invention is directed to an intravascular stent that has a pattern or configuration that permits the stent to be tightly compressed or crimped onto a catheter to provide an extremely low profile and to prevent relative movement between the stent and the catheter. The stent also is highly flexible along its longitudinal axis to facilitate delivery through tortuous body lumens, but which is stiff and stable enough radially in its expanded condition to maintain the patency of a body lumen such as an artery when the stent is implanted therein. Importantly, the stent pattern provides for excellent column strength in the event the stent is exposed to longitudinal compressive forces.

The stent of the present invention generally includes a plurality of cylindrical rings that are interconnected to form the stent. The stent typically is mounted on a balloon catheter if it is balloon expandable or mounted on or in a catheter without a balloon if it is self-expanding.

Each of the cylindrical rings making up the stent have a proximal end and a distal end and a cylindrical plane defined by a cylindrical outer wall surface that extends circumferentially between the proximal end and the distal end of the cylindrical ring. Generally the cylindrical rings have a serpentine or undulating shape which includes at least one U-shaped element, and typically each ring has more than one U-shaped element. The cylindrical rings are interconnected by at least two links which attach one cylindrical ring to an adjacent cylindrical ring.

The at least two links between adjacent rings are configured to enhance longitudinal stability, including axial column strength when exposed to longitudinal compressive forces, yet provide longitudinal flexibility to navigate tortuous body lumens (e.g., coronary arteries).

The stent may be formed from a tube by laser cutting the pattern of cylindrical rings and links in the tube, or by wire-based stents having welds, both of which are well known in the art.

### BRIEF DESCRIPTION OF THE DRAWINGS

FIG. 1 is an elevational view of a prior stent depicting rings collapsing into each other or nesting as a result of a longitudinally compressive load.

FIG. 2 is an elevational view of a prior art stent depicting rings that are in phase with links connecting a peak of one ring to a valley of an adjacent ring.

FIG. 3 is an elevational view of a portion of a prior art stent depicting cylindrical rings in an out-of-phase configuration with a connection between the adjacent rings at the peak of one ring connected to the peak of an adjacent ring.

FIG. 4 is an elevational view of a portion of a prior art stent in which adjacent rings are connected by links that extend from the mid-strut or bar arm of one ring to the mid-strut or bar arm of adjacent rings.

FIG. 5 is an elevational view of a portion of a prior art stent depicting off-set peak-to-peak rings in which the peaks of adjacent rings are offset circumferentially and links extend from the peak of one ring to the peak of an adjacent ring.

FIG. 6A is an elevational view of a test fixture in which a prior art stent is mounted on a mandrel for eventual longitudinal compression.

FIG. 6B is an elevational view of the test apparatus of FIG. 6A in which the prior stent is subjected to longitudinal compressive loading thereby collapsing the stent rings into a nesting configuration.

FIG. 7 is a graph depicting data taken from the test apparatus of FIGS. 6A and 6B in which prior art stents were subjected to longitudinal compressive loading.

FIG. 8A is an elevational view of a portion of a prior art stent depicting an offset peak-to-peak stent pattern in which there are two connecting links between adjacent rings where the ends of the link connect adjacent peaks.

FIG. 8B is an elevational view of a portion of the prior art stent of FIG. 8A depicting stent rings collapsing into each other as a result of longitudinal compressive forces on the stent.

FIG. 9A is an elevational view of a finite element analysis simulation of a stent having an offset peak-to-peak stent pattern with two connecting links between adjacent rings.

FIG. 9B is an elevational view of the stent of FIG. 9A depicting a finite element analysis simulation of the stent being compressed longitudinally so that adjacent rings nest one within another.

FIG. 10 is a partial elevational view in which a peak of one ring is connected by a link to the mid-bar arm of an adjacent ring.

FIG. 11 is an elevational view of a portion of a stent in which the links connecting peaks of adjacent rings are substantially wider than other portions of the stent.

FIG. 12 is an elevational view of a portion of a stent in which peaks of adjacent rings are connected by links that are substantially wider than other portions of the stent.

FIG. 13 is an elevational view of a portion of a stent in which adjacent rings are connected by links extending into bar arms all of which are substantially wider than other portions of the stent.

FIG. 14 is an elevational view of a portion of a stent in which some links and some bar arms are substantially wider than other portions of the stent.

FIG. 15 is an elevational view of a portion of a stent depicting at least three links connecting adjacent rings of the stent structure.

FIG. 16 is an elevational view of a portion of a stent depicting two links between adjacent rings in close proximity to each other with a third link connecting the same adjacent rings but approximately 180° from the two links.

FIG. 17 is an elevational view of a portion of a stent depicting a three-two-three link alternating configuration.

FIG. 18 is an elevational view of a portion of a stent depicting three connecting links connecting several rings at the ends of the stent to the adjacent body rings and two connecting links between adjacent body rings.

FIG. 19 is an elevational view of a portion of a stent depicting the peaks of one ring bending in a first direction and the adjacent peaks of an adjacent ring bending in a second, opposite direction.

FIG. 20A is an elevational view of a portion of a stent in which a peak on one ring has a shortened height as compared to other peaks and is adjacent to a peak on an adjacent ring that is bent or offset.

FIG. 20B is an elevational view of a portion of a stent depicting at least one peak of a ring being shorter than other peaks in the ring and adjacent to a bent peak on an adjacent ring.

FIG. 20C is an elevational view of a portion of a stent depicting multiple peaks on adjacent rings that are shorter in length than other peaks and being adjacent to bent peaks on an adjacent ring.

FIG. 21 is an elevational view of a portion of a stent in which two links between adjacent rings have a substantially longer length.

FIG. 22 is an elevational view of a portion of a stent depicting certain adjacent rings connected by links having a length shorter than the links connecting other adjacent rings.

FIG. 23 is an elevational view of a portion of a stent depicting rings connected by links having at least two bends and a straight portion.

FIG. 24 is an elevational view of a portion of a stent depicting a first link having a first angulation connecting adjacent rings and a second link having a second, different angulation connecting the same adjacent rings.

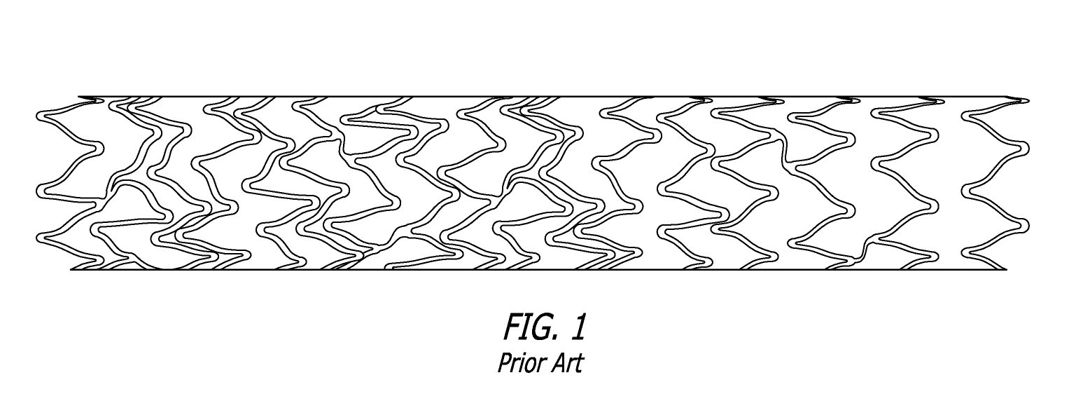

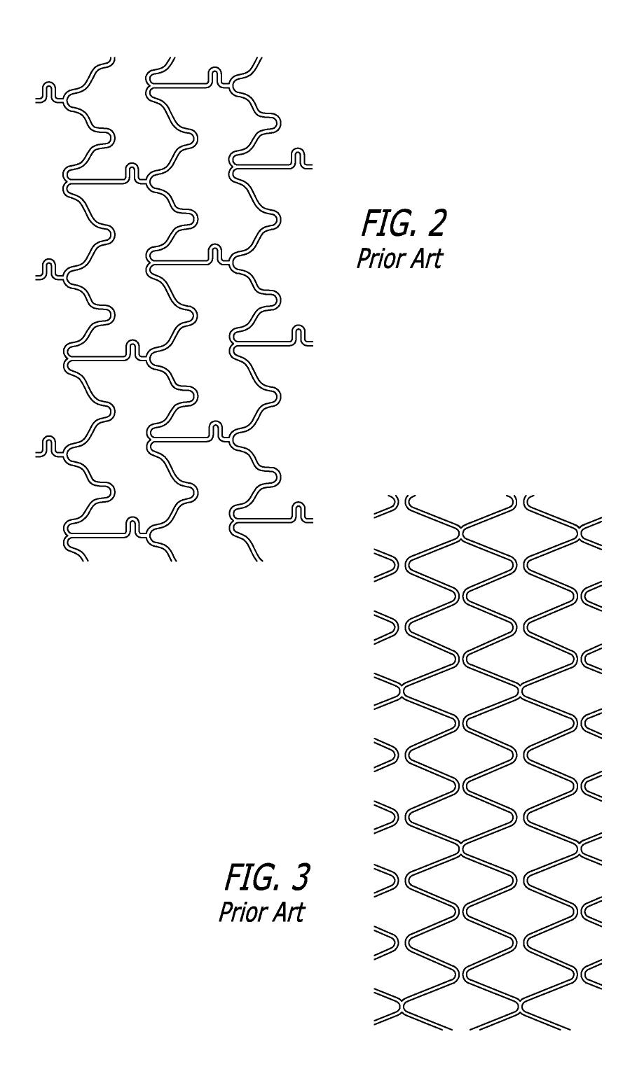

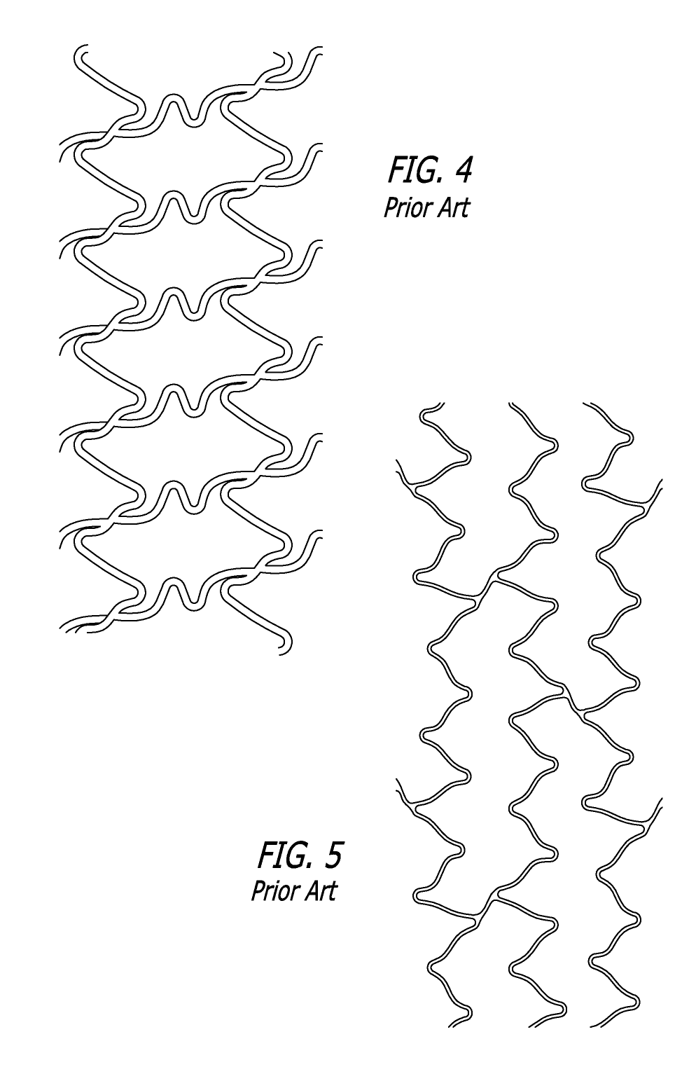

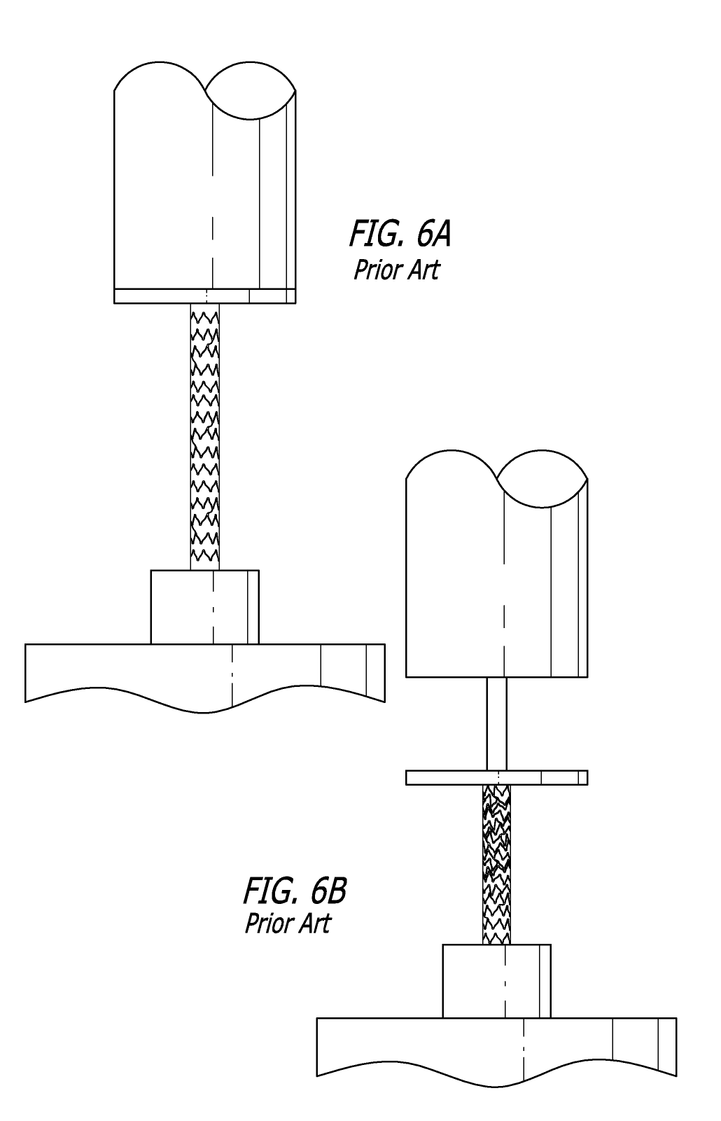

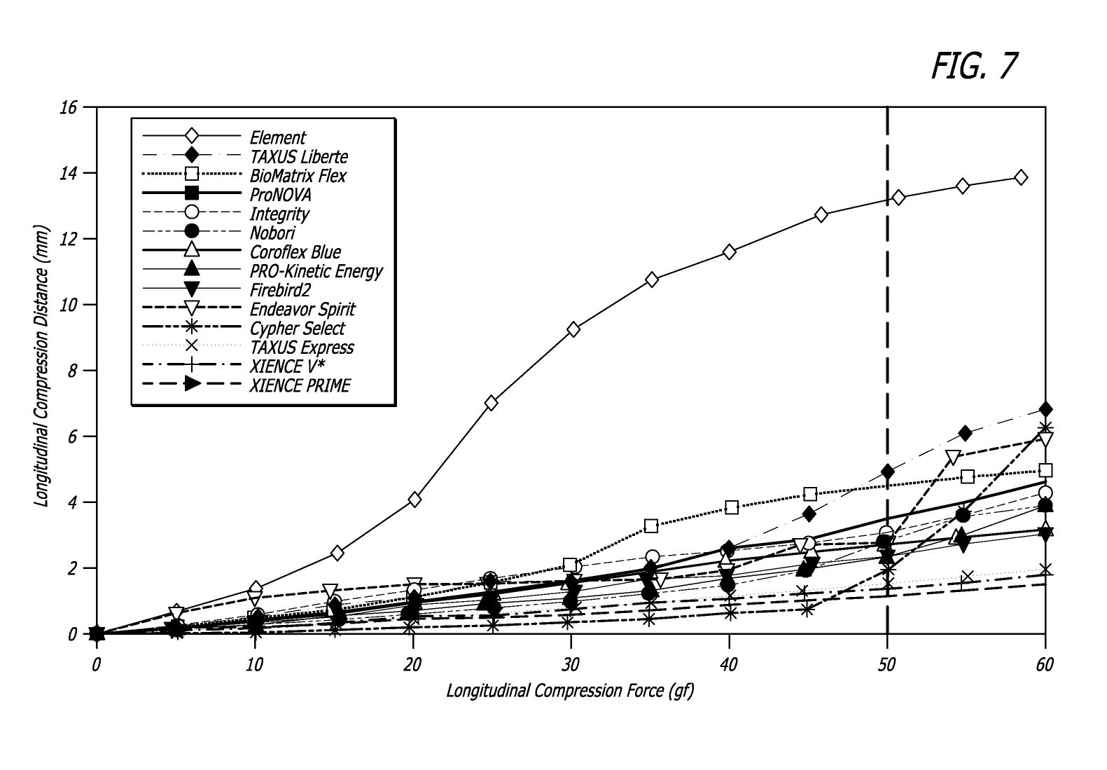

### DETAILED DESCRIPTION OF THE PREFERRED EMBODIMENTS

A novel stent platform is presented to facilitate improved resistance to longitudinal compression when compared to conventional two-link offset peak-to-peak stent patterns. Longitudinal compression has emerged in the literature and in technical research as a stent attribute which portrays the stability of an implanted stent structure under longitudinal compressive loads. These loads may be imparted on an implanted stent by guide catheter impact (ostial lesions), device pullback (IVUS, etc.), or when crossing an implanted stent with another stent. It has been shown that insufficient resistance to longitudinal compression may relate to excessive stent shortening after implantation, which has potential undesired implications for drug eluting stent (DES) safety and efficacy.

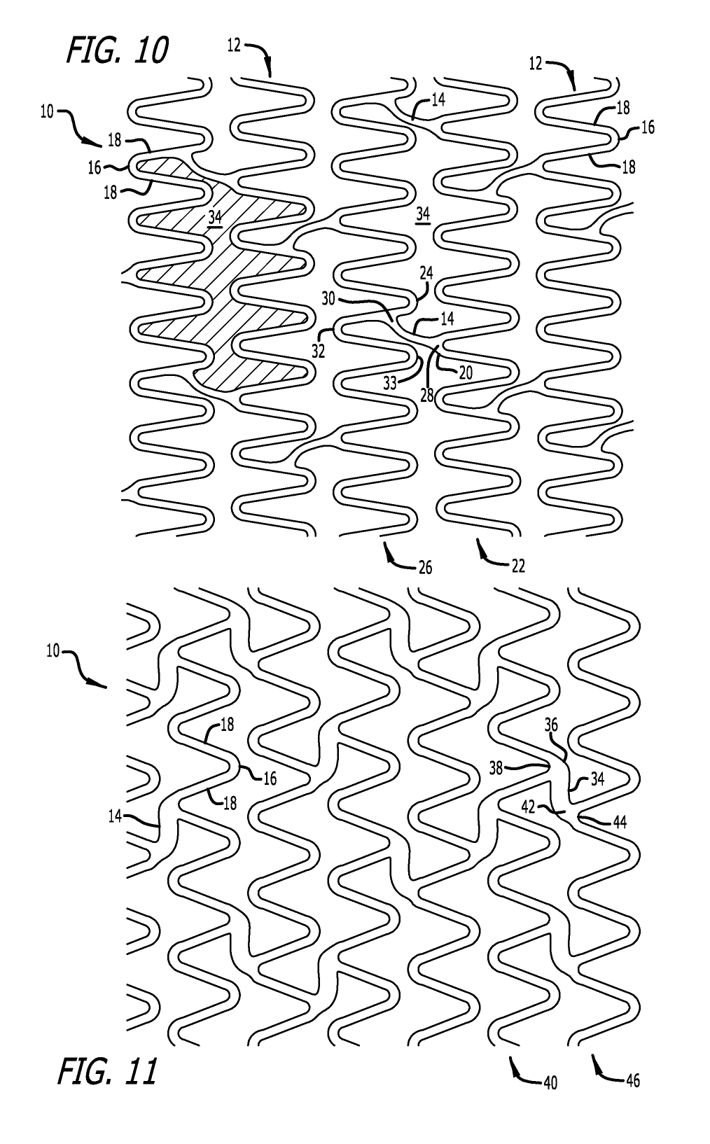

Referring to FIGS. 10-24, the various embodiments of the stents disclosed herein have some common structural features. Typically, the stent 10 includes a plurality of cylindrical rings 12 that are interconnected by links 14. The stents are in a tubular form, however, for ease of viewing, are shown flattened. The stents are usually laser cut from a tubular member so there are no discrete parts, but it is beneficial for identification to refer to various parts such as the rings and links. The cylindrical rings generally have undulations or peaks 16 that are connected by bar arms 18. Alternatively, the same stent pattern can be formed by bending a wire and welding the cross-points or overlapping struts.

One important aspect of the stent embodiment disclosed herein, is to balance the longitudinal flexibility of the stent (for delivery through tortuous body lumens such as the coronary arteries) with longitudinal stability including the ability to withstand longitudinal compression during delivery of the stent to a body lumen.

In one embodiment, as shown in FIG. 10, longitudinal stability is accomplished through the inclusion of peak to mid-bar arm links to minimize the offset peak-to-peak stent pattern and bring it closer to a peak-to-peak design. The peaks 16 are offset circumferentially so that they are neither out-of-phase (all peaks pointing toward each other), nor in-phase (all peaks pointing in the same direction and longitudinally aligned). In other words, first peak 20 of first ring 22 points towards second peak 24 of second ring 26, but the first peak 20 is circumferentially offset from second peak 24. The first ring 22 is connected to adjacent second ring 26 by link 14 which has a first end 28 connected to first peak 20 and a second end 30 connected to bar arm 18 about mid-way between second peak 24 and third peak 32. By incorporating peak-to-mid bar arm links instead of offset peak-to-peak links, the offset between adjacent rings is minimized. This structure reduces the offset between adjacent rings such that the stent cannot nest as easily as the conventional two-link offset peak-to-peak stent pattern under similar longitudinally compressive loads. Because the stent does not nest as much, rings adjacent to each other will come into contact sooner, minimizing longitudinal compression. Further, the peak to mid-bar arm links not only encourage early contact and reduced nesting propensity, the design fundamentally reduces the moment arm or cantilever response of the structure under longitudinal compressive loading.

As shown in FIG. 10, the peak to mid-strut links configuration for stent cell 34 prevents rings from collapsing during longitudinal compression of the deployed stent. Peak to mid-strut links also help to prevent full nesting of an offset peak-to-peak-type stent pattern (out-of-phase) and thus reduce longitudinal collapse due to strut contact during longitudinal loading.

Further, this embodiment presents an effort to manage the major tradeoff (longitudinal instability) of a two-link offset peak-to-peak stent design while retaining the major benefits (radial strength, stiffness and flexibility) of the stent pattern shown in FIG. 10. The peak-to-mid bar arm pattern provides a more structurally efficient and stable deployed stent, which in turns leads to a stent pattern with optimal clinical performance with respect to deliverability, safety and efficacy.

The following stent platform benefits are a result of providing the peak-to-mid bar arm stent embodiment (FIG. 10) as compared to a two-link offset peak-to-peak stent structure (e.g., FIG. 5).
(1) Improved design stability as a result of a reduced moment arm or reduced cantilever effect during longitudinal compression;
(2) Increased propensity for protective and stabilizing ring-to-ring contact during longitudinal stent compression;
(3) Potential for thinner strut design with maintained longitudinal stability; and
(4) Potential improved safety and efficacy under longitudinally compressive circumstances.

The peak-to-mid bar arm stent pattern of FIG. 10 may be applied to stent designs intended for any body lumen, including but not limited to coronary and peripheral blood vessels. While the benefits described are intended specifically for balloon expandable coronary stents, improved longitudinal stability may be broadly beneficial for the stenting of any body lumen with any type of stent deployment mechanism. The described embodiment demonstrates potential improvements over two-link offset peak-to-peak stent patterns, however, the features presented may also improve stents with any number of links, including those laser cut from either metallic tubing or polymer. As set forth herein, the stent patterns of the invention can be formed by a wire-based stent having welds at the overlapping struts or abutting struts.

In another embodiment, shown in FIG. 11, an improvement in longitudinal stability is accomplished through the inclusion of a specifically widened link and link-adjacent bar arms. This improvement is intended to enhance the stent's resistance to deformation under longitudinal compression loads. By specifically widening these portions of the stent structure, the two-link stent structure more effectively can resist longitudinal compression loads. With this benefit provided, a two-link, thin-strut stent platform with increased flexibility could be created without trading off longitudinal stability.

As shown in FIG. 11, the links 14 have a width dimension and a radial thickness dimension, as do the peaks 16 and bar arms 18. As an example, the radial thickness of the various structural elements of the stent can range from 0.020 inch to 0.0038 inch, and either have uniform thickness throughout the stent, or portions of the stent can be radially thicker than others. Similarly, the width of the structural elements typically can range from 0.024 inch to 0.0041 inch, and can be of uniform width or different elements can have different widths. In this embodiment, the width of links 34 is substantially greater than the width of conventional links and can range from 0.025 inch to 0.0074 inch. Widened link 34 has a first end 36 connected to a first peak 38 on a first ring 40 and a second end 42 connected to a second peak 44 on an adjacent second ring 46. The widened link structure 34 is designed to be anywhere from 5% to 80% wider than any of the widths of the peaks, bar arms, or other elements comprising the stent pattern. In one embodiment, the wider links 34 are from 20% to 40% wider than any other of the stent structural elements. In another embodiment, the range is from 40% to 65% wider. This widened link structure 34 is intended to provide greater resistance to longitudinal compression when compared to standard uniform stent patterns. As described, compressive loads may occur due to proximal guide catheter contact with the deployed stents, IVUS pullback contact, and catching on stent struts (or similar device), or during stent crossing.

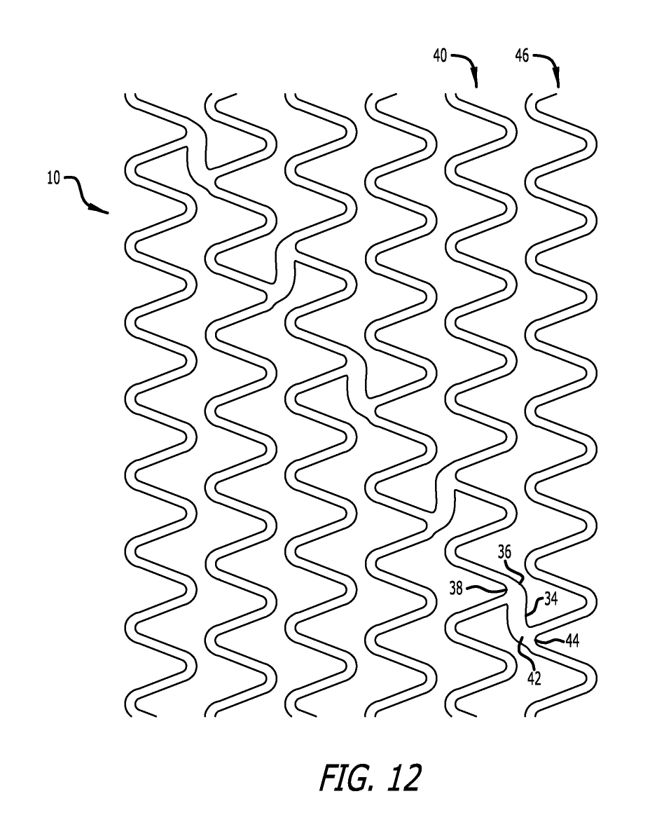

This widened structure may be included at all link locations, or placed selectively throughout a portion of the link regions within the stent pattern. Two different link patterns are shown in FIGS. 11 and 12.

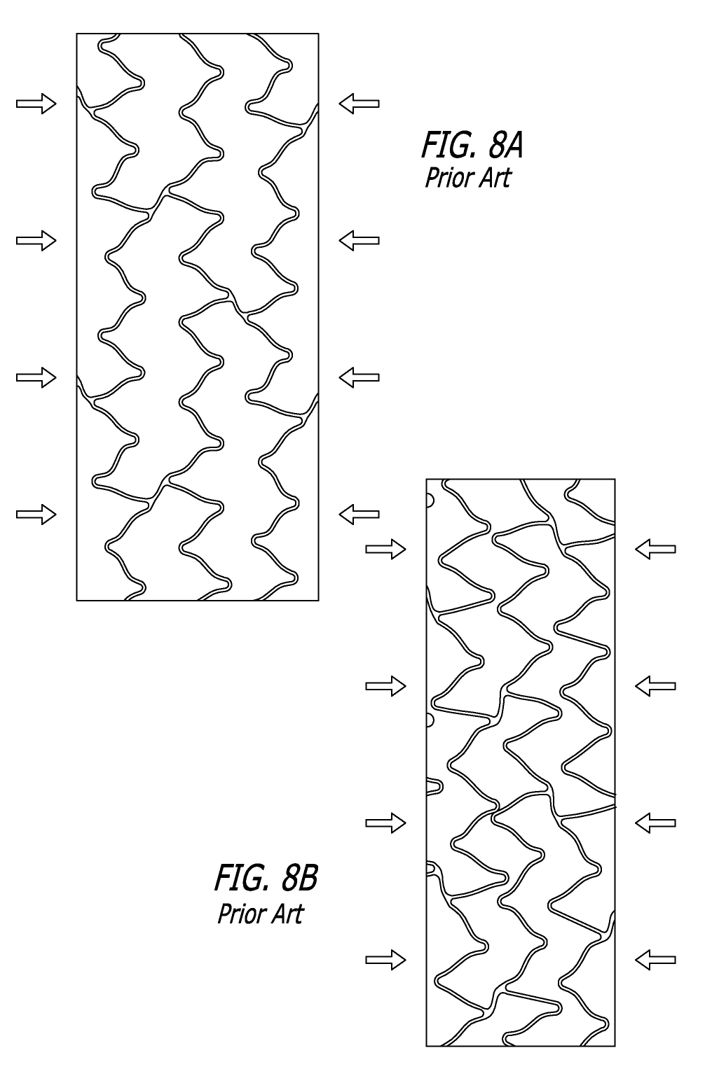

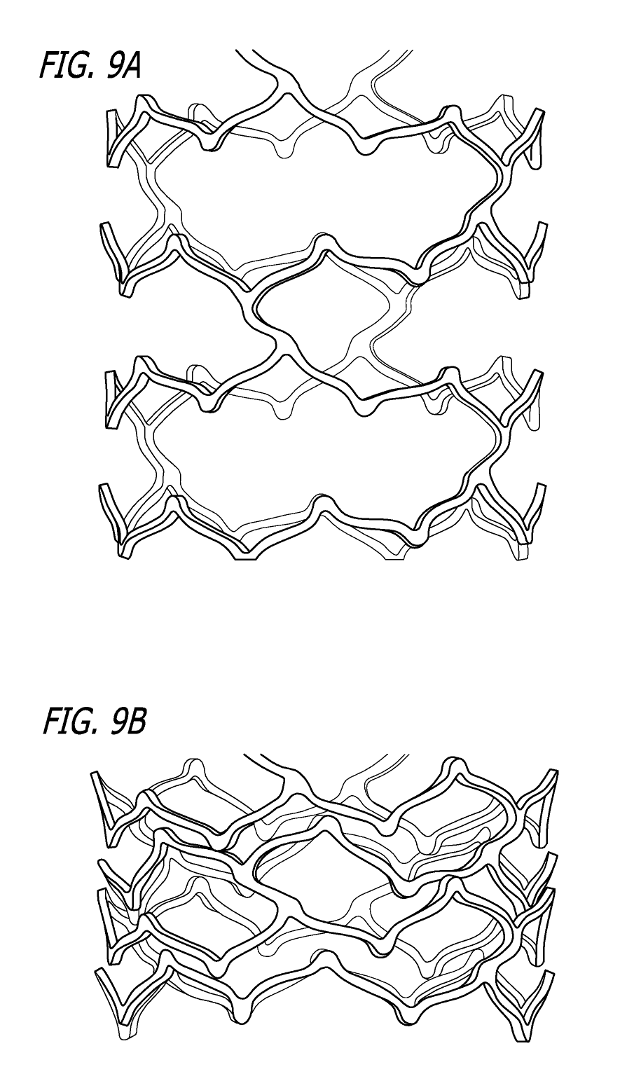

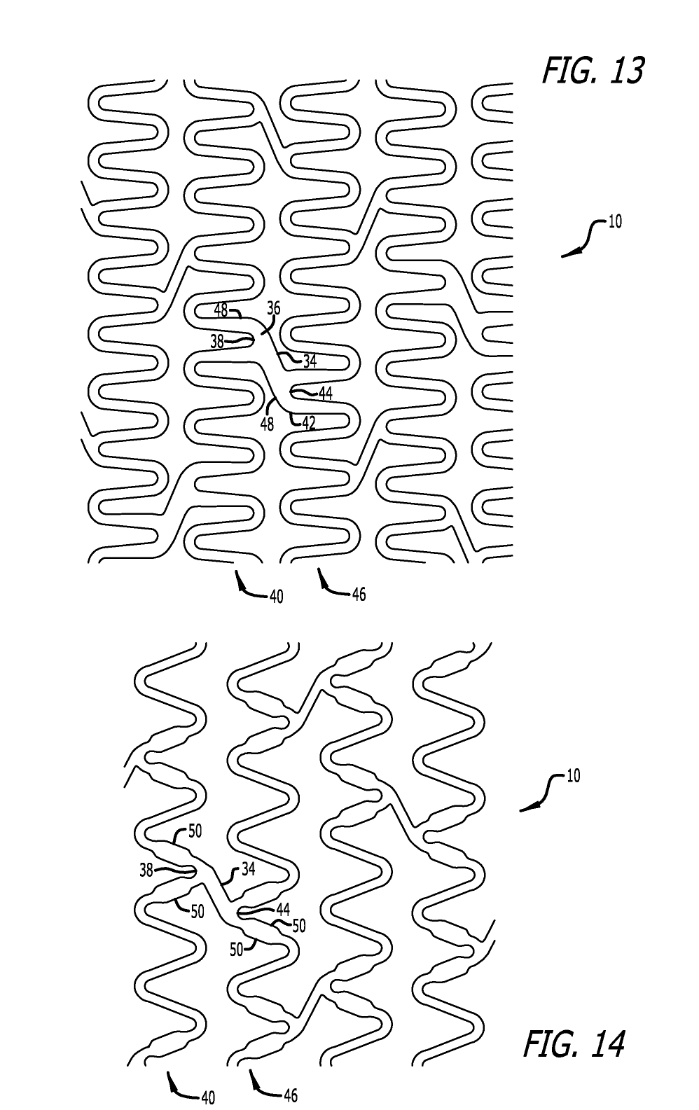

In addition to widening the link structure of the stent, it has been shown previously that the bar arm structure adjacent to the links incurs substantial bending stress during longitudinal compression (FIGS. 8A-8B and 9A-9B). To produce a structure that more efficiently distributes loads during longitudinal compression, two more design embodiments are disclosed in FIGS. 13 and 14. FIG. 13 shows a specifically widened link 34 and a widened link-adjacent bar arm 48, while FIG. 14 shows a selectively widened link-adjacent bar arm 50. As shown previously by the deformed stent structure, bar arm bending substantially occurs in link-adjacent bar arms.

In FIGS. 11-14, four stent embodiments are presented to manage the major tradeoff (longitudinal instability) of a two-link offset peak-to-peak stent pattern while retaining the major benefits (radial strength, stiffness and flexibility) of this stent pattern. Through these design features, and any combination of the four structural improvements described, a more structurally efficient and stable deployed stent can be produced, which in turn leads to a design with optimal clinical performance with respect to deliverability, safety and efficacy.

The embodiments shown in FIGS. 11-14 can be modified to provide the following alternative embodiments:
(a) The inclusion of selectively widened link features to produce stable longitudinal compression behavior;
(b) Spiral or alternating patterning using selectively wider link sections throughout a portion of the stent structure, or universally throughout the structure;
(c) The inclusion of a single widened link for each ring pair to serve as a structural reinforcement against longitudinal compression, with a narrower link included to maintain stent flexibility;
(d) The inclusion of selectively widened link and link-adjacent bar arm features to improve longitudinal stability of the stent;
(e) The inclusion of selectively widened link-adjacent bar arm features to improve longitudinal stability of the stent; and
(f) Any combination of the embodiments (a) through (e).

The following stent platform benefits are a result of incorporating the widened link 34 embodiments into a two-link offset peak-to-peak stent structure:
(1) Improved design stability during longitudinal compression;
(2) Improved uniformity of stent deformation when subjected to longitudinal compressive loads;
(3) Increased propensity for protective and stabilizing ring-to-ring contact during longitudinal stent compression;
(4) Potential for thinner strut design (thinner bar arms and peaks) with maintained structural stability;
(5) Potential for improved safety and efficacy under longitudinally compressive circumstances; and
(6) A wider link structure potentially produces a more fracture resistant design (stent pieces have a lower potential to fracture completely or separate).

In another embodiment, an improvement in longitudinal stability is accomplished through the incorporation of additional links connecting adjacent rings of the stent. By increasing the number of links between two adjacent rings from two links to three links, the stent structure can more effectively resist longitudinal compression by limiting the amount of ring nesting that occurs as a result of an applied longitudinal compressive load. The construction of these additional links may be present throughout the length of the stent, or the geometry may be tailored to increase the number of links only at the end rings, or staggered throughout the length of the stent. The location of the additional links can be optimized to increase longitudinal stability while maintaining adequate stent flexibility necessary for deliverability and conformability.

Shown below in FIG. 15, three links 52 connect two adjacent rings 51A,51B along the length of the stent 10 to prevent rings from collapsing during longitudinal compression of the deployed stent. The inclusion of three links limits the amount of nesting that can occur between adjacent rings during application of a longitudinal load by decreasing the number of free peaks 54 (peaks that do not connect to the adjacent ring) per cell. The cell geometry 56 along the circumference of the stent can be either uniform (equal number of free peaks in every cell), or uneven (uneven number of free peaks in every cell) as shown in FIG. 15. The direction of the links 52 can either be positioned in the alternating orientations (as shown in FIG. 15) or in the same orientation along the length of the stent. The patterning of the orientation along the length of the stent can be optimized for stent flexibility.

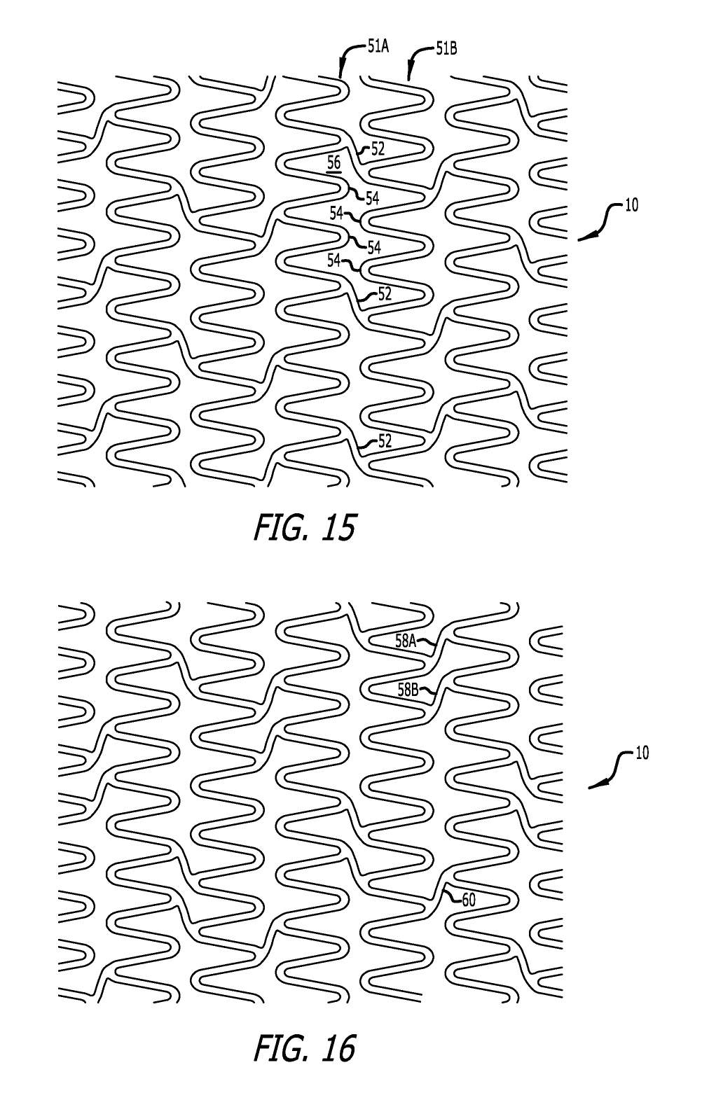

The configuration of a three-link pattern 52 of FIG. 15 can be modified to optimize stent flexibility while minimizing the amount of stent shortening during longitudinal compression. FIG. 16 illustrates one configuration in which first and second links 58A,58B of the three links are placed on adjacent crests of a ring to provide axial stability, while third link 60 is placed at approximately 180° separated from the first and second links 58A,58B to provide sufficient stent flexibility. It is known that two-link patterns are more flexible than three-link patterns. This design configuration incorporates third link 60 to provide longitudinal stability while behaving more similarly to a two-link design with respect to stent flexibility since two links (the first and second links 58A,58B) are positioned close together on adjacent crests.

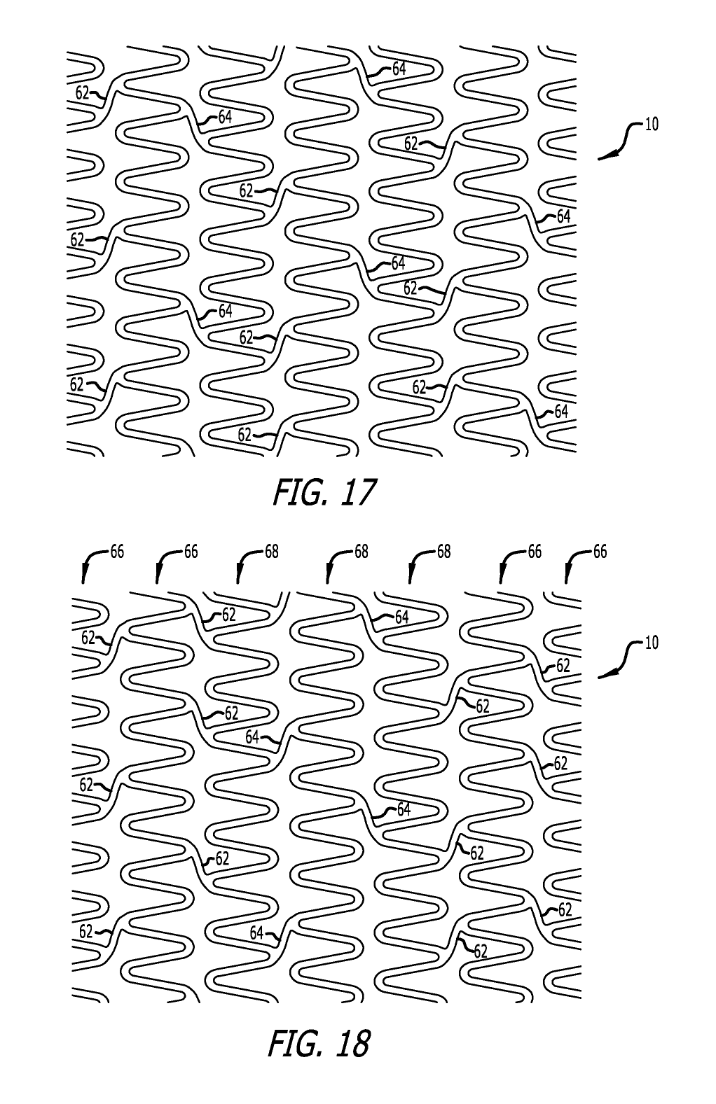

FIG. 17 illustrates another embodiment of alternating three-two-three-link configurations. This stent pattern (which can be in various combinations of three links 62 and two links 64 such as three-two-two-three, three-three-two-three-three-two, etc.) provides stable areas along the stent length which minimize stent shortening (three-link locations), while providing adequate stent flexibility (two-link locations). The positioning of these links could be optimized for stent flexibility, longitudinal stability and sufficient scaffolding. The orientation of these links may be alternating, as shown in FIG. 18, or in the same direction.

In order to provide stability to the ends of the stent 10, which most likely experience the majority of longitudinal instability since this is the most common location for secondary products to come in contact with the expanded stent, three links 62 connect the first few end rings 66 of the stent. To maintain stent flexibility, the body rings 68 of the stent are connected by two links. The number of end rings that contain three links can be optimized for each stent length as necessary to maintain sufficient stent flexibility. The link orientation may be alternating, as shown in FIG. 17, or in the same direction.

The following stent platform benefits are a result of incorporating the three-link embodiments into a two-link offset peak-to-peak stent structure.
(1) Improved design stability during longitudinal compression.
(2) Increased propensity for protective and stabilizing ring-to-ring contact during longitudinal stent compression.
(3) Potential for thinner strut design with maintained structural stability.
(4) Potential for improved safety and efficacy under longitudinally compressive circumstances.

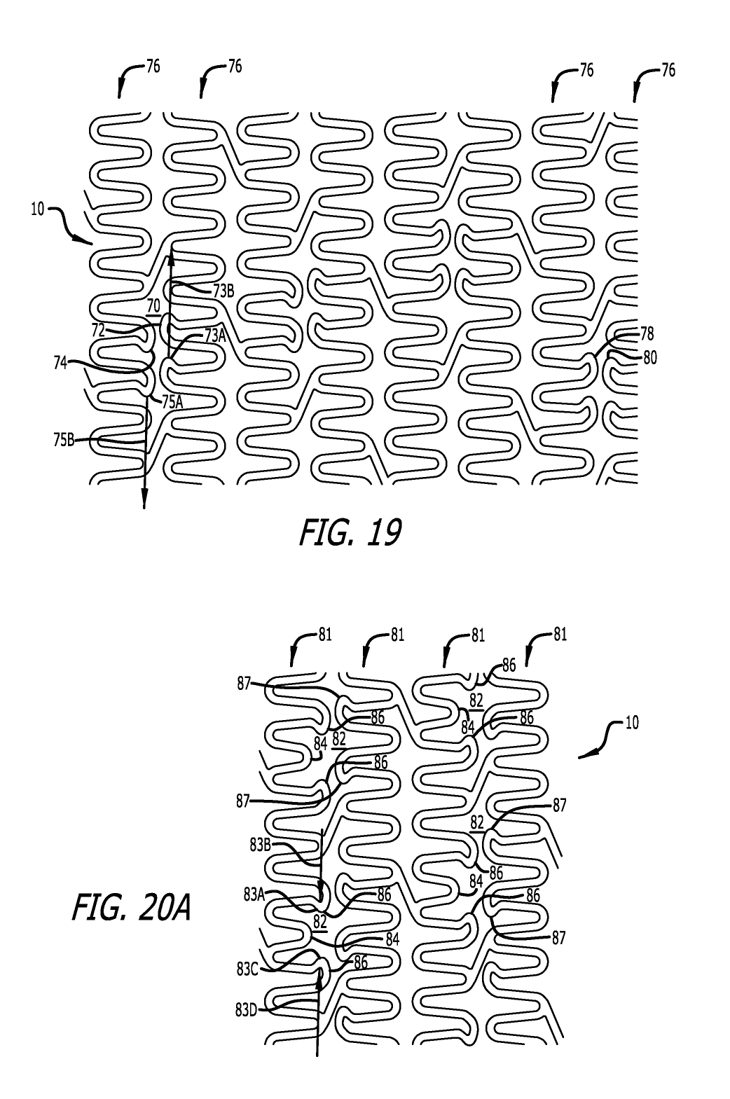

In another embodiment, as shown in FIG. 19, the improvement in longitudinal stability is accomplished through the inclusion of specifically oriented or turned peaks which are designed to come into contact with each other during stent longitudinal compression. By reacting longitudinal loads between rings through this peak-to-peak contact, the two-link stent structure more effectively can resist longitudinal compression loads. With this benefit provided a two-link, thin-strut stent platform with inherent increased flexibility could be created without trading off longitudinal stability.

As shown in FIG. 19, stent 10 has a stent cell 70 that provides a cell design in which a first peak 72 is turned in a direction opposite to an adjacent second peak 74. Thus, first peak 72 has a first offset bend 73A pointing in a first direction 73B, and a second offset bend 75A pointing in a second direction 75B that is opposite to the first direction 73B. This configuration allows for nesting of the rings 76 during crimping while also producing an expanded stent structure that encourages ring-to-ring contact during longitudinal compression. Under longitudinal compressive loads, the first peak 72 and the first offset bend 73A will come into contact with the second peak 74 and the second offset bend 75A, thereby resisting any further axial shortening of stent 10 as the rings 76 move toward each other. Through this ring-to-ring contact, the stent structure's ability to resist longitudinal compression is enhanced in situations when longitudinal loads are imparted on the stent. As described earlier, these loads may occur due to proximal guide catheter contact with the deployed stent, IVUS pullback contact and catching on stent struts (or similar device), or stent crossing. Alternatively, as shown in FIG. 19, third peak 78 is turned in one direction and adjacent fourth peak 80 is turned in the same direction. Under longitudinal compressive loads, the third peak 78 will come into contact with the fourth peak 80, thereby resisting any further axial shortening of stent 10 as the rings 76 move toward each other.

These two embodiments will manage the major tradeoff (longitudinal instability) of a two-link offset peak-to-peak stent design while retaining the major benefits (radial strength and stiffness, flexibility) of this stent pattern. Further, these embodiments, and any combination thereof, provide a more structurally efficient and stable deployed stent, which in turn leads to a design with optimal clinical performance with respect to deliverability, safety and efficacy.

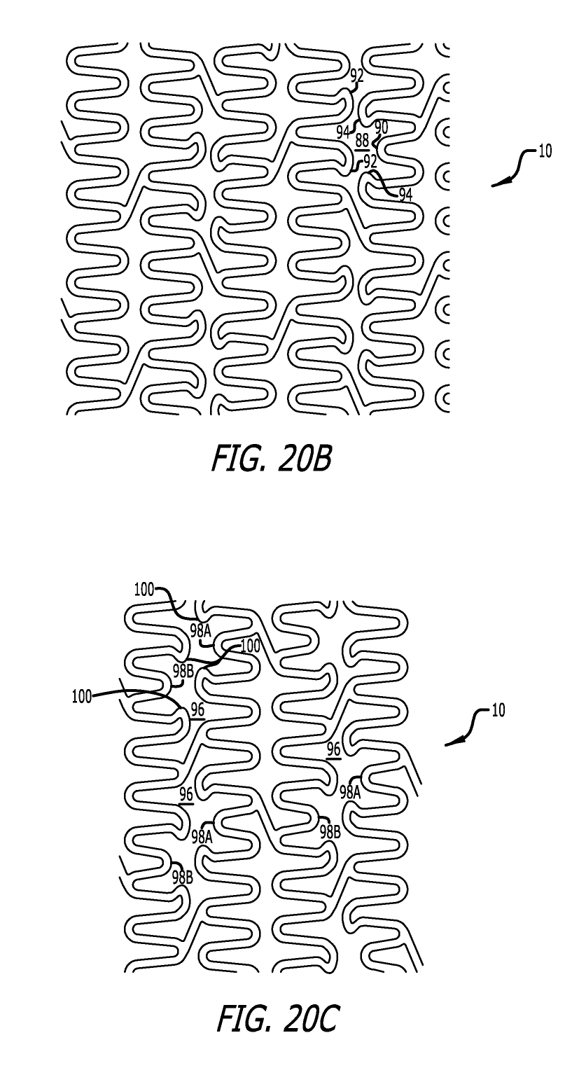

In another embodiment, shown in FIG. 20A, stent 10 has a plurality of rings 81 and a stent cell 82 which includes multiple short peaks 84 that nest between two longer adjacent stent peaks 86, which have a first offset bend 83A pointed in first direction 83B and a second offset bend 83C pointed in a second direction 83D which is opposite to first direction 83B. This nesting occurs during stent crimping, as the longer peaks 86 within a ring move toward each other. This peak configuration allows for nesting of the short peaks 84 within the long peaks 86 during crimping, while also producing an expanded stent structure that encourages ring-to-ring contact during longitudinal compression. The rings 81 have an out-of-phase configuration where the peaks 86 on one ring point toward and are somewhat longitudinally aligned with peaks 87 on an adjacent ring. The out-of-phase configuration coupled with the ring-to-ring contact, provides the stent structure's ability to resist longitudinal compression and is enhanced in situations when longitudinal loads are imparted on the stent. For example, during longitudinal compression, peaks 86 on one ring will nest into and contact peaks 87 on an adjacent ring and resist any further axial shortening As described earlier, these loads may occur due to proximal guide catheter contact with the deployed stent, IVUS pullback contact and catching on stent struts (or similar device), or stent crossing. In this embodiment (FIG. 20A), the rings 81 are connected to each other by links 85 that have an angular orientation relative to the stent longitudinal axis.

Similarly, in FIG. 20B, stent cell 88 includes a single short peak 90 placed within the stent cell. This design allows for more effective contact between expanded stent rings due to first peak 92 being turned in a direction opposite to second peak 94. As a longitudinal compressive load impacts stent 10, single short peak 90 does not interfere structurally as first peak 92 contacts second peak 94 to resist or prevent further axial shortening as the rings move toward each other.

FIG. 20C shows a symmetric cell 96 embodiment, wherein two opposing short peaks 98A,98B nest within adjacent turned peaks 100 on both sides of stent cell 96. This pattern may provide the most balanced resistance to longitudinal compression, however, any combination of the embodiments in FIGS. 20A-20C may be used throughout a stent pattern to provide sufficient longitudinal stability while allowing for a reasonable crimp profile.

The embodiments shown in FIGS. 20A-20C are presented in order to manage the major tradeoff (longitudinal instability) of a two-link offset peak-to-peak stent design while retaining the major benefits (radial strength and stiffness, flexibility) of this stent design type. Through the design feature(s), a more structurally efficient and stable deployed stent can be produced, which in turns leads to a design with optimal clinical performance with respect to deliverability, safety and efficacy. These embodiments further provide the following alternative aspects.
(a) The combination of short and long crests with turned peak crests within a stent pattern to produce stable longitudinal compression behavior.
(b) The nesting or optimal closing of a short crest and adjacent turned peak crests.
(c) The inclusion of turned peak crests with short bar arms to induce ring-to-ring contact during stent longitudinal compression.
(d) The inclusion of turned peak crests within a stent pattern to produce stable longitudinal compression behavior.
(e) The inclusion of multiple turned-peak configurations to induce optimal ring-to-ring contact during stent longitudinal compression at a variety of expansion diameters.

The following stent platform benefits are a result of incorporating the FIGS. 20A-20C embodiments into a two-link offset peak-to-peak stent structure.
(1) Improved design stability during longitudinal compression.
(2) Increased propensity for protective and stabilizing ring-to-ring contact during longitudinal stent compression.
(3) Potential for thinner strut design with maintained structural stability.
(4) Potential for improved safety and efficacy under longitudinally compressive circumstances.

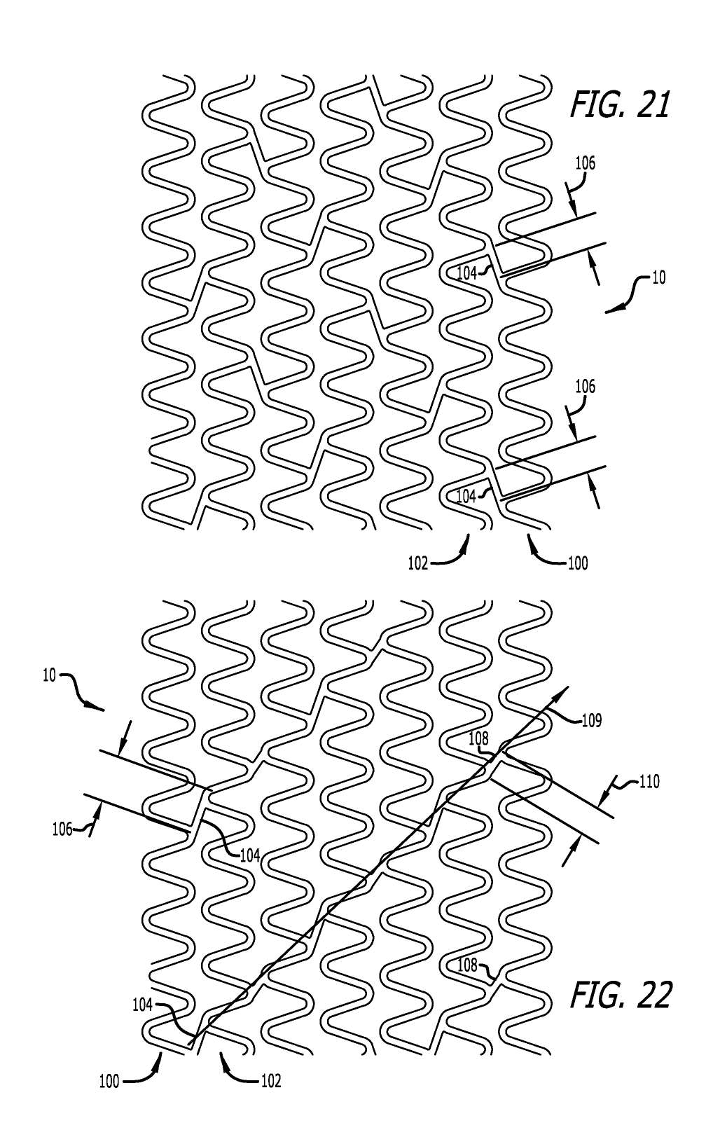

In order for adjacent rings (first and second rings 100,102) in an offset peak-to-peak stent 10 to be connected, the links 104 would need to be angulated as shown in FIG. 21. To make the stent flexible, these angulated links 104 have a first length 106, so they can easily swing to facilitate delivery of the stent to the lesion site. However, they also easily swing and result in excessive shortening when the implanted stent 10 experiences a longitudinal compressive load. To minimize this effect, FIG. 22 shows a stent 10 with long and short links which are alternated from one ring to the next. Thus, links 104 have a first length 106 that is longer than links 108 having a short length 110. These short and long links are positioned in a spiral pattern 109 to provide a spinal structure in the longitudinal direction of the stent. Unlike the long links, the short links do not swing as easily. The stent in FIG. 22, as a whole, is more effective at resisting a longitudinal compressive load than is the stent of FIG. 21, while flexibility is not significantly affected.

The following stent platform benefits in the FIGS. 21-22 embodiments are as follows.
(1) Improved design stability during longitudinal compression.
(2) Increased propensity for protective and stabilizing ring-to-ring contact during longitudinal stent compression.
(3) Potential for thinner strut design with maintained longitudinal stability.
(4) Potential for improved safety and efficacy under longitudinally compressive circumstances.

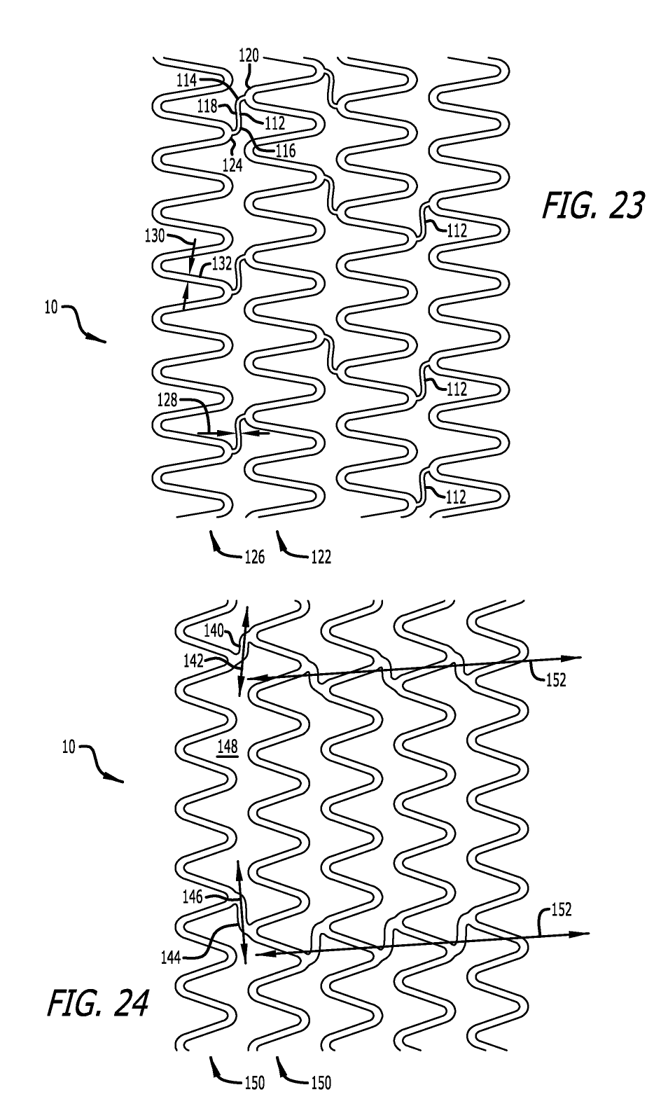

In the embodiment of FIG. 23, the improvement in longitudinal stability is accomplished through the incorporation of additional links connecting adjacent rings of the stent (i.e., more than two links between adjacent rings). By increasing the number of links between two adjacent rings, the stent structure can more effectively resist longitudinal compression by limiting the amount of ring nesting that occurs as a result of applied longitudinal load. The construction of these additional links may be present throughout the length of the stent, or the geometry may be tailored to increase the number of links only at the end rings, or staggered throughout the length of the stent. The location of the additional links can be optimized to reduce longitudinal stability while maintaining adequate stent flexibility necessary for deliverability and conformability.

The stent 10 shown in FIG. 23 includes three links 112 between adjacent rings, the links having at least a first bend 114, a second bend 116, and straight portion 118 therebetween. The links 112 connect first peak 120 on first ring 122 to adjacent second peak 124 on second ring 126. The three links 112 also have a width 128 that can be up to 65% less than a width 130 of the bar arms 132. Typically, peak-to-peak stent designs incorporate two links connecting adjacent rings to provide the required flexibility/deliverability. By incorporating three links in a peak-to-peak design, the flexibility of the design may be compromised. In an offset peak-to-peak stent design, one concern is longitudinal stability. A two-link offset peak-to-peak design may have compromised longitudinal stability, which may be improved by incorporating an additional link. In order to maintain stent flexibility with the additional link, links 112, as shown in FIG. 23, having first and second bends 116,118 are incorporated into the design. The three-link aspect of the design minimizes the amount of nesting of adjacent rings while the curvy, narrow links 112 provide stent flexibility. This type of link configuration can be incorporated throughout the length of the stent, or may be integrated in certain regions of the stent, such as the end rings or the end ring regions (multiple rings at the ends of the stent).

The following stent platform benefits are a result of incorporating the curvy links 112 into a two-link offset peak to peak stent structure.
(1) Increased propensity for protective and stabilizing ring-to-ring contact during longitudinal stent compression.
(2) Increased propensity for protective and stabilizing ring-to-ring contact during longitudinal stent compression.
(3) Potential for thinner strut design with maintained longitudinal stability.
(4) Potential for improved safety and efficacy under longitudinally compressive circumstances.

In an embodiment shown in FIG. 24, the improvement in longitudinal stability is accomplished through the inclusion of alternating angled link orientations. By including opposite link directions between adjacent stent rings, the two-link stent structure more effectively resists longitudinal compression loads instead of swinging. Additionally, spiral patterning is also proposed to more effectively transfer compressive loads between stent rings. With these benefits provided, a two-link, thin-strut stent platform with increased flexibility could be created without trading off longitudinal stability.

Shown in FIG. 24, first links 140 are angled in a first direction 142 and second links 144 are angled in a second direction 146 within a stent cell 148 to prevent rings 150 from collapsing during longitudinal compression of the deployed stent 10. The oppositely oriented links 140,144 are therefore both present in a single stent cell 148 to increase resistance to the ring rotating and nesting within the adjacent ring. These link connections may be grouped throughout part or all of the stent structure in an aligned fashion 152 along the length of the stent to increase longitudinal stability. These mini spines 152 may increase longitudinal stability.

The embodiment of FIG. 24 can be modified to include alternative embodiments.
(a) The combination of oppositely angled links within a stent cell produces stable longitudinal compression behavior.
(b) Alternating orientation of links within a stent cell prevents nesting of rings as the stent is longitudinally compressed.
(c) The inclusion of repeating links in alignment with adjacent links, spanning the length of the stent partially or completely.

While the invention has been illustrated and described herein, in terms of its use as an intravascular stent, it will be apparent to those skilled in the art that the stent can be used in other body lumens. Further, particular sizes and dimensions, number of undulations or peaks per ring, materials used, and the like have been described herein and are provided as examples only. Other modifications and improvements may be made without departing from the scope of the invention.

What is claimed:

1. A stent, comprising:
a tubular member having a plurality of rings connected by links, the rings having short peaks and long peaks;
adjacent rings being out of phase wherein the long peaks of one ring point toward the long peaks of an adjacent ring; and
the short peaks on one ring being located between long peaks on the same ring in order to promote ring-to-ring contact when longitudinally compressive loads are imparted on the stent.

2. The stent of claim 1, wherein the long peaks have offset bends at the peaks.

3. The stent of claim 2, wherein a first long peak has a first offset bend and a second long peak has a second offset bend.

4. The stent of claim 3, wherein the first offset bend points in first direction and the second offset bend points in a second direction opposite to the first direction.

5. The stent of claim 4, wherein the first offset bend points toward the second offset bend.

6. The stent of claim 5, wherein a short peak is positioned between the first offset bend and the second offset bend.

7. The stent of claim 1, wherein adjacent rings are connected by two links.

8. The stent of claim 7, wherein the links connect a long peak on one ring with a long peak on an adjacent ring.

9. The stent of claim 8, wherein the links have an angular orientation relative to a longitudinal axis of the stent.

10. The stent of claim 1, wherein the stent is balloon expandable or self-expanding.

11. A stent, comprising:
a tubular member having a plurality of rings connected by links, the rings having short peaks and long peaks;
a plurality of stent cells having multiple short peaks on adjacent rings;
adjacent rings being out of phase wherein the long peaks of one ring point toward the long peaks of an adjacent ring; and
the short peaks on one ring being located between long peaks on the same ring in order to promote ring-to-ring contact when longitudinally compressive loads are imparted on the stent.

12. The stent of claim 11, wherein the long peaks have offset bends at the peaks.

13. The stent of claim 12, wherein a first long peak has a first offset bend and a second long peak has a second offset bend.

14. The stent of claim 13, wherein the first offset bend points in first direction and the second offset bend points in a second direction opposite to the first direction.

15. The stent of claim 14, wherein the first offset bend points toward the second offset bend.

16. The stent of claim 5, wherein a short peak is positioned between the first offset bend and the second offset bend.

17. The stent of claim 11, wherein adjacent rings are connected by two links.

18. The stent of claim 17, wherein the links connect a long peak on one ring with a long peak on an adjacent ring.

19. The stent of claim 18, wherein the links have an angular orientation relative to a longitudinal axis of the stent.

20. The stent of claim 11, wherein the stent is balloon expandable or self-expanding.
